(19) United States
(12) Patent Application Publication
(10) Pub. No.: US 2008/0071344 A1
(43) Pub. Date: Mar. 20, 2008

(54) MEDICAL DEVICE WITH POROUS SURFACE

(75) Inventors: Kevin Silberg, Big Lake, MN (US); Jeffrey S. Lindquist, Maple Grove, MN (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 11/850,140

(22) Filed: Sep. 5, 2007

Related U.S. Application Data
(60) Provisional application No. 60/825,965, filed on Sep. 18, 2006.

(51) Int. Cl. A61F 2/84; A61F 2/82; A61F 2/86

(52) U.S. Cl. 623/1.11; 623/1.2; 623/1.39; 623/1.42; 623/1.51

(57) ABSTRACT

Medical devices, such as endoprostheses, and methods of making the devices are described. In some implementations, the endoprostheses is a stent having a tubular body with an outer wall surface, and an inner wall surface defining a stent central lumen. One or more regions of the outer wall surface and the inner wall surfaces is formed by a porous, sintered metal layer. One or more regions of the porous, sintered metal layer provides a porous reservoir or media for drug material. The porous, sintered metal layer in one or more regions of the inner wall surface provides relatively decreased friction, increased hardness and lower tack, as compared to excipient polymeric coating material for stents, and are positioned to facilitate improved, relatively lower resistance withdrawal of a delivery balloon from the stent central lumen.

### CROSS-REFERENCE TO RELATED APPLICATIONS

This application claims priority under 35 USC §119(e) to U.S. Provisional Patent Application Ser. No. 60/825,965, filed on Sep. 18, 2006, the entire contents of which are hereby incorporated by reference herein.

### TECHNICAL FIELD

The invention relates to medical devices, such as endoprostheses (e.g., stents).

### BACKGROUND

The body defines various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, or allowed to expand, into contact with the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

### SUMMARY

The invention relates to medical devices, such as endoprostheses.

According to one aspect of the invention, a medical device in the form of a stent has a tubular body with an outer wall surface, and an inner wall surface defining a stent central lumen, with one or more regions of the outer and inner wall surfaces being formed by a porous, sintered metal layer. The porous, sintered metal layer provides a porous reservoir or media for drug material, and provides relatively reduced friction, increased hardness and lower tack, as compared to excipient polymeric coating material for stents, the one or more regions of porous, sintered metal layer being positioned to facilitate improved device tracking and relatively lower resistance to withdrawal of a stent delivery device from the stent central lumen.

Implementations of this aspect of the invention may include one or more of the following additional features. The porous, sintered metal layer in one or more regions comprises a porous, sintered metal coating. Preferably, the porous, sintered metal coating comprise a very thin, porous, sintered metal coating, e.g., with a thickness in the range of about 5 micron to about 50 micron. The very thin, porous, sintered metal coating is bonded to the surface of the tubular metal body of the stent. The porous, sintered metal forms the tubular metal body of the stent. The tubular metal body of the stent is formed of woven wire. The tubular metal body is formed of porous, sintered metal mesh.

According to another aspect of the invention, a method for introducing a medical device in the form of a stent into a lumen of a patient's body includes the steps of: mounting a stent delivery device within a stent central lumen, the stent having a tubular body with an outer wall surface, and an inner wall surface defining the stent central lumen, with one or more regions of the outer wall surface and the and inner wall surface formed of a porous, sintered metal layer, the stent as mounted disposed in a condition having a first outer diameter; at a site of delivery of the stent within the lumen of the patient's body, acting to enlarge the stent to a second, relatively larger outer diameter and into engagement with surrounding surfaces of the lumen of the patient's body; and withdrawing the stent delivery device from the stent central lumen, the porous, sintered metal coating of one or more regions of the outer wall surface and the inner wall surface providing relatively reduced friction, increased hardness and lower tack, as compared to excipient polymeric coating material for stents, facilitating improved device tracking and relatively lower resistance to withdrawal of the stent delivery device from the stent central lumen.

Implementations of this aspect of the invention may include the following additional features. The porous, sintered metal layer of one or more regions of the outer wall surface and the inner wall surface provides a porous reservoir or media for drug material, and the method comprises the further step of delivering the drug material from the porous reservoir or media into the lumen of the patient's body at the site of delivery. The stent delivery device is a balloon catheter, and the method further comprises expanding the catheter balloon within the stent central lumen to cause the stent to enlarge to a second, relatively larger outer diameter and into engagement with surrounding surfaces of the lumen of the patient's body. The stent is self-expanding, and the method further comprises releasing the stent from the stent delivery device to allow the stent to enlarge to a second, relatively larger outer diameter and into engagement with surrounding surfaces of the lumen of the patient's body.

Implementations may also include one or more of the following advantages. The implantable stent drug delivery system provides improved frictional, hardness, tack and drug delivery properties for improved device tracking, lower resistance to balloon withdrawal, and improved diffusion of drug, resulting in improved SDS delivery and complete drug release, and possibly, although not yet proven, improved or faster neointimal growth (endothelialization) resulting in improved healing.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification will control. In addition, the materials, methods, and examples are illustrative only and not intended to be limiting. Other features and advantages of the invention will be apparent from the following detailed description, and from the claims.

### DESCRIPTION OF DRAWINGS

The FIGURE is a perspective view of an implementation of an expanded stent.

### DETAILED DESCRIPTION

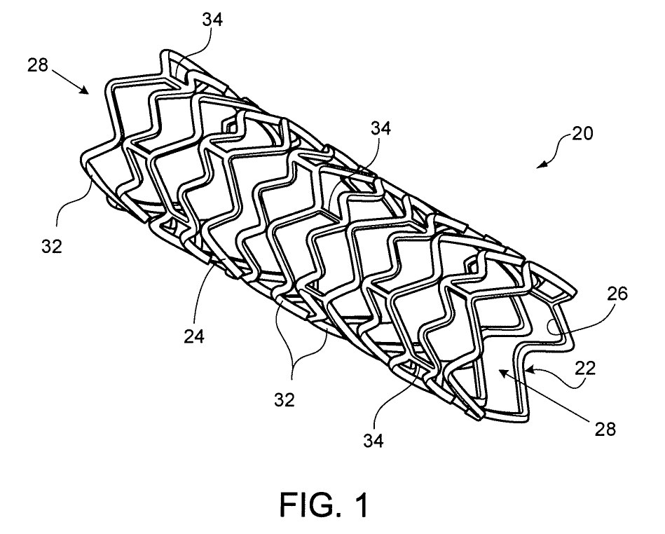

Referring to FIGURE 1, a stent 20 has the form of a tubular body 22 defining an outer wall surface 24 and an inner wall surface 26. The inner wall surface defines a central lumen 28. The stent tubular body member 22 is formed by a plurality of bands 32 and a plurality of connectors 34 that extend between and connect adjacent bands. During use, bands 32 are expanded from an initial, small outer diameter to a relatively larger outer diameter to contact the outer wall surface 24 of stent 20 against a surrounding wall of a vessel, thereby maintaining the patency of the vessel. Connectors 34 provide stent 20 with flexibility and conformability that allow the stent to adapt to the contours of the vessel.

Stent 20 can include (e.g., be manufactured from) one or more biocompatible materials with mechanical properties that allow a stent including a composite material to be compacted, and subsequently expanded to support a vessel. In some implementations, stent 20 can have an ultimate tensile yield strength (YS) of about 20-150 ksi, greater than about 15% elongation to failure, and a modulus of elasticity of about 10-60 msi. When stent 20 is expanded, the material can be stretched to strains on the order of about 0.3. Examples of suitable materials for the tubular body of stent 20 include stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6Al-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2993, and entitled “Medical Devices and Methods of Making Same;” and U.S. application Ser. No. 11/035,316, filed Jan. 3, 2005, and entitled “Medical Devices and Methods of Making Same.” Other materials include elastic biocompatible metals such as a superelastic or pseudo-elastic metal alloy, as described, for example, in Schetsky, L. McDonald, “Shape Memory Alloys,” Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

In some implementations, the tubular metal body 22 forming stent 20 includes one or more materials that enhance visibility by MRI. Examples of MRI materials include non-ferrous metals (e.g., copper, silver, platinum, or gold) and non-ferrous metal-alloys containing paramagnetic elements (e.g., dysprosium or gadolinium) such as terbium-dysprosium, dysprosium, and gadolinium. Alternatively or additionally, the metallic matrix can include one or more materials having low magnetic susceptibility to reduce magnetic susceptibility artifacts, which during imaging can interfere with imaging of tissue, e.g., adjacent to and/or surrounding the stent. Low magnetic susceptibility materials include those described above, such as tantalum, platinum, titanium, niobium, copper, and alloys containing these elements.

The bands 32 and connectors 34 defining the tubular metal body 22 of the stent 20 are formed, as shown, by cutting the tube. Selected portions of the tube can be removed to form bands 32 and connectors 34 by laser cutting, as described in Saunders U.S. Pat. No. 5,780,807. In certain implementations, during laser cutting, a liquid carrier, such as a solvent or an oil, may be flowed through the lumen of the tube. The carrier can prevent dross formed on one portion of the tube from re-depositing on another portion, and/or reduce formation of recast material on the tube. Other methods of removing portions of the tube can be used, such as mechanical machining (e.g., micro-machining), electrical discharge machining (EDM), and photoetching (e.g., acid photoetching).

As an example, while stent 20 is described above as being formed wholly of composite material, in other implementations, the composite material forms one or more selected portions of the medical device. For example, stent 20 can include multiple layers in which one or more layers include a composite material, and one or more layers do not include a composite material. The layer or layers including a composite material can include the same composite material or different composite materials. The layer or layers not including a composite material may include one or more of the biocompatible matrix materials listed above. The layering of the composite material provides yet another way to tailor and tune the properties of the medical device. Stents including multiple layers are described, for example, in U.S. Patent Publication No. 2004-0044397 and in Heath U.S. Pat. No. 6,287,331.

In some implementations, after bands 32 and connectors 34 are formed, areas of the tube affected by the cutting operation above can be removed. For example, laser machining of bands 32 and connectors 34 can leave a surface layer of melted and resolidified material and/or oxidized metal that can adversely affect the mechanical properties and performance of stent 20. The affected areas can be removed mechanically (such as by grit blasting or honing) and/or chemically (such as by etching or electropolishing). In some implementations, the tubular member can be near net size and configuration at this stage. “Near-net size” means that the tube has a relatively thin envelope of material that is next removed to provide a semi-finished stent, e.g. for receiving the porous, sintered metal coating to be bonded to the surface, as discussed below. In some implementations, the tube is formed less than about 25% oversized, e.g., less than about 15%, 10%, or 5% oversized.

The unfinished stent is then finished to form stent 20. Since the unfinished stent can be formed to near-net size, relatively little of the unfinished stent must be removed to finish the stent. As a result, further processing (which could damage the stent) and discard of costly materials can be reduced. In some implementations, about 0.0001 inch of the stent material can be removed by chemical milling and/or electropolishing to yield a semi-finished stent.

Stent 20 can be of a desired shape and size (e.g., coronary stents, aortic stents, peripheral vascular stents, gastrointestinal stents, urology stents, and neurology stents). Depending on the intended application, stent 20 can have an outer diameter of between, for example, about 1 mm to about 46 mm. In certain implementations, a coronary stent can have an expanded outer diameter of from about 2 mm to about 6 mm. In some implementations, a peripheral stent can have an expanded outer diameter of from about 5 mm to about 24 mm. In certain implementations, a gastrointestinal and/or urology stent can have an expanded outer diameter of from about 6 mm to about 30 mm. In some implementations, a neurology stent can have an expanded outer diameter of from about 1 mm to about 12 mm. An abdominal aortic aneurysm (AAA) stent and a thoracic aortic aneurysm (TAA) stent can have an outer diameter from about 20 mm to about 46 mm. Stent 20 can be balloon-expandable, self-expandable, or a combination of both (e.g., Andersen et al. U.S. Pat. No. 5,366,504).

Also, current, conventional, block copolymer-based implantable stent drug delivery technology utilizes a 16.5 mole % polystyrene, linear, triblock, styrenic polymer system, commonly referred to as SIBS, as the excipient material. With current, known paclitaxel/SIBS stent coatings, the excipient material is soft, elastomeric, and possesses some inherent tack. These inherent properties of SIBS provide excellent elastic recovery and resistance to fatigue in stent regions of high strain but may result in low occurrence instances of resistance to balloon withdrawal after the BE stent is deployed. Resistance to withdrawal is being demonstrated to be a key factor in DE stent delivery. The very thin, porous, sintered metal coating of the outer wall surface and the inner wall surface of the stent 20 addresses these issues.

In one particular implementation, the improved stent 20 of the FIGURE is provided with a non-polymeric, very thin, porous sintered metal coating, e.g. with thickness in the range of about 5 micron to about 50 micron, bonded to one or more regions of the outer wall surface and the inner wall surface of the stent to provide a porous reservoir, or media, for drug material. This thin, porous, sintered metal material can be manipulated in terms of density, porosity, e.g. down to 2 micron size, or tortuosity, to control drug elution rates and duration. In other implementations, the stent 20 may be a seamless stent produced entirely from sintered metal, sintered mesh, woven wire, etc.

In particular, the described implantable stent drug delivery system provides improved frictional, hardness, tack and drug delivery properties for lower resistance to balloon withdrawal and improved diffusion of drug, resulting in improved SDS delivery and complete drug release.

Porous sintered metal powders, fibers, or wires are utilized in many industries as very high performance, complex, filter material of virtually any shape with near-exact dimensional tolerances. Furnace sintering is an established metallurgical method of bonding every contact point of very small metal species to produce strong, porous, ductile laminates or material objects with porosity down to 2 micron size.

The porous reservoir formed by the sinter metal coating or body of the stent 20 preferably includes a releasable therapeutic agent, drug, or a pharmaceutically active compound, such as described in U.S. Pat. No. 5,674,242, U.S. application Ser. No. 09/895,415, filed Jul. 2, 2001, and U.S. application Ser. No. 10/232,265, filed Aug. 30, 2002. The therapeutic agents, drugs, or pharmaceutically active compounds can include, for example, anti-thrombogenic agents, antioxidants, anti-inflammatory agents, anesthetic agents, anti-coagulants, and antibiotics.

In current, conventional SIBS-based stent drug delivery technology employing known paclitaxel/SIBS stent coatings, the drug exposed on the surface of the excipient coating is quickly solubilized into the tissue during the initial stage of drug release. This initial “spike” or “burst” of release constitutes a substantial portion of the total cumulative device drug release, while a large portion of the total drug content remains within the coating for extended periods of time. The ability to control release kinetics and to provide complete drug release may be linked to late successful healing and resistance to thrombosis.

In use, stent 20 can be employed, e.g., delivered and expanded, using a catheter delivery system. Catheter systems are described in, for example, Wang U.S. Pat. No. 5,195,969, Hamlin U.S. Pat. No. 5,270,086, and Raeder-Devens U.S. Pat. No. 6,726,712. Stents and stent delivery are also exemplified by the Radius® or Symbiot® systems, available from Boston Scientific Scimed, Maple Grove, Minn.

### Other Embodiments

While a number of implementations have been described above, the invention is not so limited. For example, in some implementations, stent 20 can be formed by fabricating a wire including the composite material, and knitting and/or weaving the wire into a tubular member. The composite materials described herein can also be used to form other medical devices.

Other implementations are within the claims.

What is claimed is:

1. A medical device, comprising a stent having a tubular body with an outer wall surface, and an inner wall surface defining a stent central lumen, with one or more regions of the outer wall surface and the inner wall surface formed by a porous, sintered metal layer,
the porous, sintered metal layer of one or more regions of the outer wall surface and the inner wall surface providing a porous reservoir or media for drug material, and
the porous, sintered metal layer of one or more regions of the inner wall surface providing relatively decreased friction, increased hardness and lower tack, as compared to excipient polymeric coating material for stents, the one or more regions of the outer wall surface and the inner wall surface being positioned to facilitate improved device tracking and relatively lower resistance withdrawal of a stent delivery device from the stent central lumen.

2. The medical device of claim 1, wherein the porous, sintered metal layer in one or more regions comprises a porous, sintered metal coating.

3. The medical device of claim 2, wherein the porous sintered metal coating comprise a very thin, porous, sintered metal coating.

4. The medical device of claim 3, wherein the very thin, porous, sintered metal coating has a thickness in the range of about 5 micron to about 50 micron.

5. The medical device of claim 3, wherein the very thin, porous, sintered metal coating is bonded to the surface of the tubular metal body of the stent.

6. The medical device of claim 1, wherein the porous, sintered metal forms the tubular metal body of the stent.

7. The medical device of claim 1, wherein the tubular metal body is formed of woven wire.

8. The medical device of claim 1, wherein the tubular metal body is formed of porous sintered metal mesh.

9. A method for introducing a medical device comprising a stent into a lumen of a patient's body, said method comprises the steps of:
mounting a stent delivery device within a stent central lumen, the stent having a tubular body with an outer wall surface, and an inner wall surface defining the stent central lumen, with one or more regions of the outer wall surface and the inner wall surface formed of a porous, sintered metal layer, the stent as mounted disposed in a condition having a first outer diameter;
at a site of delivery of the stent within the lumen of the patient's body, acting to enlarge the stent to a second, relatively larger outer diameter and into engagement with surrounding surfaces of the lumen of the patient's body; and
withdrawing the stent delivery device from the stent central lumen, the porous, sintered metal coating of one or more regions of the outer wall surface and the inner wall surface providing relatively reduced friction, increased hardness and lower tack, as compared to excipient polymeric coating material for stents, facilitating improved device tracking and relatively lower resistance to withdrawal of the stent delivery device from the stent central lumen.

10. The method of claim 9, wherein the porous, sintered metal layer of one or more regions of the outer wall surface and the inner wall surface provides a porous reservoir or media for drug material, and the method comprises the further step of delivering the drug material from the porous reservoir or media into the lumen of the patient's body at the site of delivery.

11. The method of claim 9, wherein the stent delivery device is a balloon catheter, and the method further comprises expanding the catheter balloon within the stent central lumen to cause the stent to enlarge to a second, relatively larger outer diameter and into engagement with surrounding surfaces of the lumen of the patient's body.

12. The method of claim 9, wherein the stent is self-expanding, and the method further comprises releasing the stent from the stent delivery device to allow the stent to enlarge to a second, relatively larger outer diameter and into engagement with surrounding surfaces of the lumen of the patient's body.
